(19) United States
(12) Patent Application Publication
(10) Pub. No.: US 2025/0127486 A1
(43) Pub. Date: Apr. 24, 2025

(54) PORTABLE VASCULAR DOPPLER SYSTEM

(71) Applicants: Oscar M. Gonzalez, Edinburg, TX (US); Jose Andres Gonzalez, McAllen, TX (US)

(72) Inventors: Oscar M. Gonzalez, Edinburg, TX (US); Jose Andres Gonzalez, McAllen, TX (US)

(21) Appl. No.: 18/489,689

(22) Filed: Oct. 18, 2023

(51) Int. Cl. A61B 8/00; A61B 8/06; A61B 8/08; G16H 10/60

(52) U.S. Cl. CPC A61B 8/4427; A61B 8/06; A61B 8/463; A61B 8/488; A61B 8/5207; A61B 8/5223; A61B 8/565; G16H 10/60

(57) ABSTRACT

In an example, a compact Doppler ultrasound probe includes an ultrasonic transducer, a wireless communication interface, and a power supply coupled to the ultrasonic transducer and the wireless communication interface. The ultrasonic transducer is configured to generate and emit ultrasound signals into a patient, receive reflected ultrasound signals from the patient, and convert the reflected ultrasound signals into electrical signals.

### FIELD

The embodiments discussed herein are related to a portable vascular doppler system.

### BACKGROUND

Unless otherwise indicated herein, the materials described herein are not prior art to the claims in the present application and are not admitted to be prior art by inclusion in this section.

Vascular Doppler ultrasound may be used to assess blood flow. For example, Vascular Doppler ultrasound systems insonate a patient's blood vessel with high frequency sound. Echoes, e.g., reflected ultrasound signals, from the patient's blood vessel are received by a transducer, digitized and processed for audio and visual waveform presentation. The resultant signals are relied upon to assess the condition of blood flow, identify stenosis and abnormalities and detect the presence of clots and aneurysms. Vascular Doppler ultrasound is risk free and pain free.

Evaluation of the vascular system is therefore very important to many medical practitioners (e.g., physicians and/or other healthcare providers). Current vascular Doppler ultrasound devices are bulky and difficult to access out of the office and even within hospital facilities or other facilities. For example, many vascular Doppler ultrasound devices are moved around a facility on a cart and have to be operated by a trained technician. A limited number of devices and/or technicians at a given facility may significantly limit the use of such devices in practice and prevent ad hoc usage (e.g., pre-op and/or post-op) by medical practitioners. Moreover, the bulk and difficulty of moving such devices may limit the willingness of medical practitioners to keep such devices on hand even if the number of devices and/or technicians is not limiting.

The subject matter claimed herein is not limited to implementations that solve any disadvantages or that operate only in environments such as those described above. Rather, this background is only provided to illustrate one example technology area where some implementations described herein may be practiced.

### SUMMARY

This Summary is provided to introduce a selection of concepts in a simplified form that are further described below in the Detailed Description. This Summary is not intended to identify key features or essential characteristics of the claimed subject matter, nor is it intended to be used as an aid in determining the scope of the claimed subject matter.

In an example embodiment, a compact Doppler ultrasound probe includes an ultrasonic transducer, a wireless communication interface, and a power supply coupled to the ultrasonic transducer and the wireless communication interface. The ultrasonic transducer is configured to generate and emit ultrasound signals into a patient, receive reflected ultrasound signals from the patient, and convert the reflected ultrasound signals into electrical signals.

In another example embodiment, an ultrasound imaging system includes a compact Doppler ultrasound probe, and a computer-readable medium. The compact Doppler ultrasound probe includes an ultrasonic transducer, a wireless communication interface, and a power supply coupled to the ultrasonic transducer and the wireless communication interface. The ultrasonic transducer is configured to generate and emit ultrasound signals into a patient, receive reflected ultrasound signals from the patient, and convert the reflected ultrasound signals into electrical signals. The computer-readable medium has computer-readable instructions stored thereon that are executable by a processor to perform or control performance of operations. The operations include receiving the electrical signals from the compact Doppler ultrasound probe through the wireless communication interface. The operations include processing the electrical signals into Doppler ultrasound data that includes blood flow velocity of the patient. The operations include outputting, to a display device, a graphic that visually depicts the blood flow velocity.

In another example embodiment, a method includes generating and emitting ultrasound signals into a patient from a compact Doppler ultrasound probe. The method includes receiving reflected ultrasound signals from the patient at the compact Doppler ultrasound probe. The method includes converting the reflected ultrasound signals into electrical signals. The method includes transmitting the electrical signals wirelessly to a mobile computing device for processing into Doppler ultrasound data.

a method to measure oxygen saturation includes generating a first signal of a subject using a first optical sensor of a torso sensor device. The method includes generating a second signal of the subject using a second optical sensor of the torso sensor device. The second optical sensor is spaced apart from the first optical sensor such that the first and second signals are generated from different locations of the subject. The method includes determining which of the first or second signals is better. The method includes generating an oxygen saturation measurement of the subject based on the better signal.

Additional features and advantages of the invention will be set forth in the description which follows, and in part will be obvious from the description, or may be learned by the practice of the invention. The features and advantages of the invention may be realized and obtained by means of the instruments and combinations particularly pointed out in the appended claims. These and other features of the present invention will become more fully apparent from the following description and appended claims, or may be learned by the practice of the invention as set forth hereinafter.

### DETAILED DESCRIPTION OF SOME EXAMPLE EMBODIMENTS

The benefits of Doppler ultrasound for evaluation of peripheral arterial disease has been well-known for years. As described by Scissons et al. in 2009, for instance:
Qualitative assessment of Doppler waveforms has been used extensively in diagnostic sonography laboratories for detecting and localizing arterial disease of the lower extremity. The primary physiological parameters influencing waveform shape are heart rate, blood pressure, and vasomotor changes. Diastolic flow reversal has been particularly useful for differentiating normal from diseased blood vessels.
Doppler arterial waveforms of peripheral arteries traditionally have been described as normal when they are triphasic. When arterial disease begins to affect blood flow, the reflective forward flow element in late diastole disappears. As arterial disease progresses, flow reversal in early diastole becomes attenuated and is subsequently lost, with the biphasic waveform pattern becoming monophasic.
   R. Scissons and A. Comerota, Confusion of Peripheral Arterial Doppler Waveform Terminology, JDMS 25:185-194 July/August 2009 (internal citations omitted). The Scissons article is herein incorporated by reference in its entirety. Thus, medical practitioners may detect and localize arterial disease using Doppler waveforms and treat their patients accordingly.

The benefits of portable ultrasound systems have also been well-known for years. For example, U.S. Pat. No. 4,413,629, granted in 1983 and herein incorporated by reference in its entirety, discloses a portable ultrasonic Doppler system for sensing movement, such as for monitoring fetal heart rate. As described in the '629 patent, the wires of hardwired Doppler systems can become tangled and/or get in the way during a physician's examination of the patient.

Embodiments herein relate to a compact Doppler ultrasound probe (hereinafter “probe”) and an associated Doppler ultrasound computer program that may be installed on and executed by a mobile computing device of a medical practitioner or other mobile computing device. The mobile computing device may be a device that the medical practitioner already has on hand, such as a smartphone, tablet computer, or laptop computer, such that the only additional hardware the medical practitioner needs to perform Doppler ultrasound is the probe. The probe may be very compact, such as about the size of a pen or pencil, that the medical practitioner can easily carry in a shirt pocket, lab coat pocket, or otherwise with the medical practitioner. Moreover, the probe may include one or more tunable ultrasonic transducers to eliminate the need for multiple probes (e.g., one for each desired frequency) as implemented in some Doppler ultrasound systems.

The mobile computing device may generate imaging data (e.g., B-mode images, Doppler components, spectral waveforms) based on signals received from the probe. The mobile computing device may enable zooming in on any desired part of the imaging data to obtain the best imaging for evaluation by the medical practitioner.

The probe and the mobile computing device communicate wirelessly to eliminate hardwired connections that can get tangled and/or interfere with imaging a desired site on a patient.

Spectral waveforms may be characterized by the mobile computing device as triphasic, biphasic, or monophasic, and the characterization may be output to the display of the mobile computing device, such as by using a different color for each type of spectral waveform.

The probe may be placed in a sterile bag for use in operation rooms during surgery.

The Doppler ultrasound system described herein may be used during post-op rounds and/or follow up visits to obtain immediate results (e.g., immediate imaging) without waiting for a bulky Doppler ultrasound system to be wheeled in or having to visit a non-portable fixed-location Doppler ultrasound system. The Doppler ultrasound system may provide access to spectral waveforms (e.g., objective graphics of blood flow) to evaluate pre-op and post-op vascular evolution, e.g., mainly in peripheral vascular cases. The Doppler ultrasound computer program may be user-friendly and provide easy access to transfer imaging data to, e.g., a digital medical file of a patient. In some embodiments, the Doppler ultrasound system may have the ability to indicate flow change. The probe may include a rechargeable battery with any suitable charging port, such as a USB port, a USB-C port, a micro USB port, or the like. Imaging data generated by the Doppler ultrasound system and/or the signals sent by the probe to the mobile computing device may be time-stamped or otherwise have a date and time of study associated therewith. The Doppler ultrasound system may include digital noise reduction, implemented in the probe and/or the Doppler ultrasound computer program, to improve sound quality over other devices.

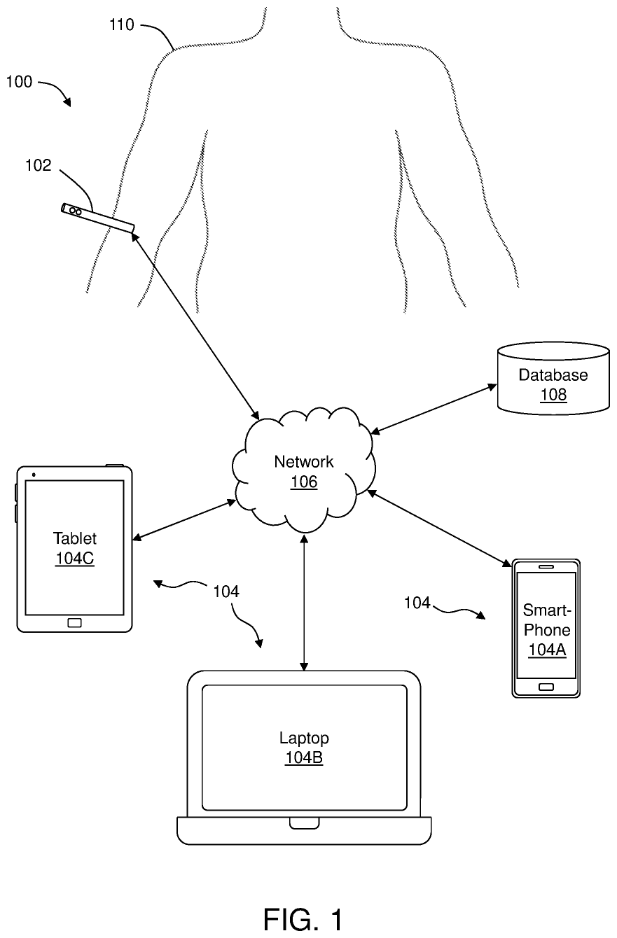

FIG. 1 is a block diagram of an example operating environment 100 of an example ultrasound imaging system, arranged in accordance with at least one embodiment described herein. The ultrasound imaging system may include a compact Doppler ultrasound probe 102 (hereinafter “probe 102”) and a Doppler ultrasound computer program (e.g., application, app, or the like) executable on any of a variety of mobile computing devices 104 or other computing devices. The environment 100 may further include one or more of a network 106, a database 108, and/or a patient 110.

The probe 102 may generally be portable and compact, such as about the size of a pencil. In some embodiments, the probe 102 has a length in a range from 5 centimeters (cm) to 20 cm, in a range from 10 cm to 17 cm, of about 15 cm, or other length. Cross-sectionally, e.g., in a plane perpendicular to the length of the probe 102 and along the entire length of the probe 102, the probe 102 may fit within a rectangular area having first and second sides, each of the first and second sides having a length in a range from 0.3 cm to 2 cm, in a range from 0.5 cm to 0.9 cm, or other length. While the cross-sectional area of the probe 102 is discussed relative to a reference “rectangular area”, the cross-sectional area of the probe 102 may have any shape (e.g., circle, ellipse, square, triangle, random, etc.) sized to fit within the reference “rectangular area”. The cross-sectional shape of the probe 102 may be consistent along the length of the probe 102; for instance, the cross-sectional shape of the probe 102 may be consistently circular along the length of the probe 102 such that the probe 102 has an overall cylindrical shape. Alternatively, the cross-sectional shape of the probe 102 may be inconsistent along the length of the probe 102.

In general, the probe 102 may be used (e.g., together with the Doppler ultrasound computer program executing on the mobile computing device 104 or other device) to image tissues and/or body fluids of a patient, such as the patient 110, and/or to image movement of tissues and body fluids. In some embodiments, data output by the probe 102 or data derived from data output by the probe 102 may include one or more of a B-mode component (e.g., showing anatomy of an imaged area of the patient), a Doppler mode component (e.g., showing blood flow direction (superimposed on the B-mode), a spectral waveform component (e.g., showing blood flow as a function of time), and/or other components.

In general, ultrasound imaging involves generating and emitting ultrasound signals into a patient (or other target), receiving reflected ultrasound signals that are reflected from internal body structures (such as tendons, muscles, joints, blood vessels, blood, and internal organs), converting the reflected ultrasound signals to electrical signals, and generating image data or other ultrasound data from the electrical signals. For example, the electrical signals may be voltage signals and B-mode images may be generated by measuring the time-varying amplitude of the voltage signals, each pixel value of a given B-mode image correlating to a corresponding measured amplitude.

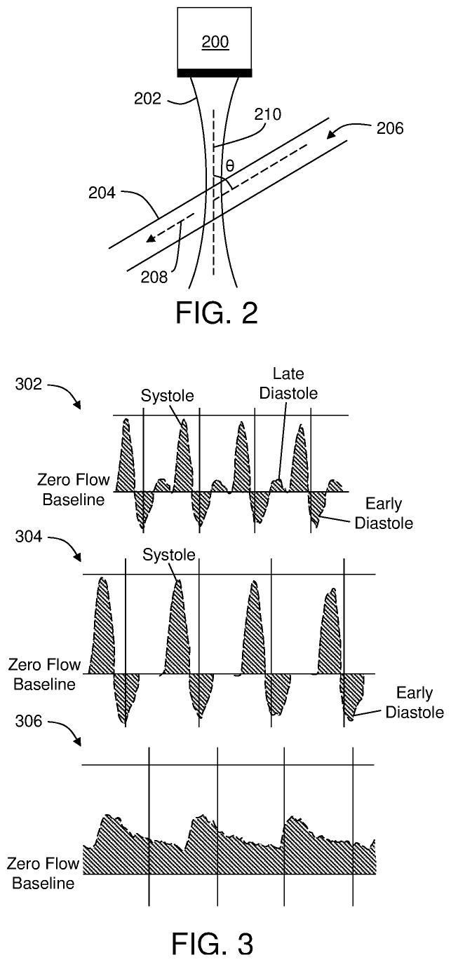

Alternatively or additionally, the Doppler effect may be employed to determine the Doppler mode component and/or the spectral waveform component using the electrical signals generated based on the reflected ultrasound signals. FIG. 2 illustrates example operation of a Doppler ultrasound probe (hereinafter “probe”) 200, arranged in accordance with at least one embodiment described herein. In operation, the probe 200 generates and emits an ultrasound signal 202 toward a blood vessel 204, the ultrasound signal 202 having a transmission frequency fₜ. Blood 206 within the blood vessel 204 flows generally in the direction indicated at 208, at least at the moment in time depicted in FIG. 2, the flow direction 208 of the blood 206 being at an angle θ relative to a direction 210 of the ultrasound signal 202. At least some of the ultrasound signal 202 is reflected by the blood 206 back toward the probe 200. Because the blood is moving, it imparts a frequency shift to the reflected ultrasound signal according to the Doppler effect. This frequency shift is referred to as the Doppler shift f_{d} and is related to the transmission frequency fₜ according to Equation 1:

$${f}_{d} = ( 2 \cdot {f}_{t} \cdot V \cdot cos θ ) / c . \qquad Equation 1$$

In equation 1, f_{d} is the Doppler shift, fₜ is the transmission frequency, V is the flow velocity of the blood 206, θ is the angle between the flow direction 208 and the ultrasound signal 202 direction 210, and c is the speed of sound in tissue. In operation, the reflected ultrasound signal having the Doppler shift f_{d} is converted to an electrical signal by the probe 200 and the electrical signal is processed (e.g., at a computing device) to determine the Doppler shift f_{d}. With f_{d}, fₜ, θ, and c being known, Equation 1 can then be used to determine the flow velocity V.

The flow direction 208 relative to the probe 200 may be determined based on, e.g., the sign of the flow velocity V (e.g., based on whether the flow velocity V is positive or negative). For example, the flow direction 208 relative to the probe 200 may be determined to be towards the probe 200 (or specifically to have a component directed towards the probe 200) if the flow velocity V is positive or away from the probe 200 (or specifically to have a component directed away from the probe 200) if the flow velocity V is negative. The flow direction 208 may be indicated in, e.g., a B-mode image of the blood vessel 204, by applying a first color (e.g., red or blue) to the blood vessel 204 image to indicate the blood flow 208 is toward the transducer 200 or a second color (e.g., blue or red) to the blood vessel 204 image to indicate the blood flow 208 is away from the transducer 200.

The spectral waveform may be determined by repeatedly calculating the flow velocity V over time (e.g., using Equation 1) and graphically depicting the flow velocity as a function of time. The flow velocity V may vary across the blood vessel 204 for any given set of measurements due to blood at different locations across the blood vessel 204 moving at different velocities such that a range of flow velocities V may be determined for any given time. The size of the range may depend on the type of blood flow. For example, blood flow within the blood vessel 204 may exhibit plug flow, laminar flow, turbulent flow, and/or other flow characteristics. In plug flow, generally exhibited by large blood vessels, the velocity of the blood flow is relatively constant (or confined to a limited range) across the blood vessel 204. In laminar flow, generally exhibited by medium blood vessels, blood within the blood vessel 204 generally follows smooth paths in layers, with each layer moving smoothly past the adjacent layers with little or no mixing such that different layers have different velocities. Turbulent flow, which is generally exhibited by small, stenotic, and/or diseased blood vessels, is characterized by chaotic changes in pressure and flow velocity across the blood vessel. The size of the range may be depicted in the spectral waveform as the thickness of the spectral waveform. Spectral broadening (e.g., thickness of the waveform) generally increases from large vessels to medium vessels to small/stenotic/diseased blood vessels.

Returning to FIG. 1, the mobile computing devices 104 may include a smartphone 104A, a laptop computer 104B, a tablet computer 104C, or other suitable mobile computing device. One or more of the mobile computing devices 104 (having stored thereon and executing the Doppler ultrasound computer program) may be used by a physician or other healthcare provider, together with the probe 102, to image the patient 110 at any desired site on the patient 110. The mobile computing devices 104 may wirelessly connect to the probe 102, e.g., via Bluetooth or other wireless networking protocol, to control the probe 102 and receive ultrasound data, such as electrical signals generated from reflected ultrasound signals. More generally, the mobile computing devices 104 may wirelessly connect to the probe 102 through the network 106.

In general, the network 106 may include one or more wide area networks (WANs) and/or local area networks (LANs) that enable the probe 102, the mobile computing devices 104, and the database 108 to communicate with each other. In some embodiments, the network 106 may include the Internet, including a global internetwork formed by logical and physical connections between multiple WANs and/or LANs. Alternately or additionally, the network 106 may include one or more cellular radio frequency (RF) networks and/or one or more wired and/or wireless networks such as 802.xx networks, Bluetooth access points, wireless access points, Internet Protocol (IP)-based networks, or other wired and/or wireless networks. The network 106 may also include servers that enable one type of network to interface with another type of network.

Ultrasound data (e.g., electrical signals generated by the probe 102 based on reflected ultrasound signals received at the probe 102)) may be received by any given one (or multiple) of the mobile computing devices 104 from the probe 102 and processed to generate ultrasound images, such as B-mode images, Doppler mode components, spectral waveforms, or the like, that may be displayed on a display of the mobile computing device 104 to the physician or other healthcare provider operating the probe 102 and the mobile computing device 104.

In some embodiments, the mobile computing device 104 executing the Doppler ultrasound computer program may further process the ultrasound data and/or the generated spectral waveforms to characterize (e.g., qualitatively assess and categorize) each spectral waveform, e.g., as monophasic, biphasic, or triphasic. For example, FIG. 3 illustrates various spectral waveforms 302, 304, 306 that may be generated, characterized, and output, e.g., by the mobile computing device 104, based on ultrasound data received from the probe 102. The spectral waveform 302 is an example of a triphasic spectral waveform. The spectral waveform 304 is an example of a biphasic spectral waveform. The spectral waveform 306 is an example of a monophasic spectral waveform. The different spectral waveform types or categories (e.g., monophasic, biphasic, triphasic) for any given patient 110 may generally depend on the condition of the patient 110 or the blood vessel being imaged at any given time. Characterization of peripheral arterial spectral waveforms (such as illustrated in FIG. 3) is basic to the diagnosis of vascular disease. Indeed, qualitative assessment of spectral waveforms such as may be generated based on the ultrasound data produced by the probe 102 has been used extensively for detecting and localizing arterial disease of the lower extremity. For example, diastolic flow reversal as exhibited by triphasic spectral waveforms such as the spectral waveform 302 has been particularly useful for differentiating normal from diseased blood vessels.

The Doppler ultrasound computer program executing on the mobile computing device 104 may implement a pattern recognition algorithm, machine learning (ML) (e.g., based on a training data set of numerous characterized spectral waveforms), and/or other processing or algorithm(s) to characterize spectral waveforms generated from the ultrasound data received from the probe 102. Alternatively or additionally, the specific characterization of a given spectral waveform may be indicated on the display of the mobile computing device 104. For example, a label of “monophasic”, “biphasic”, or “triphasic” may be displayed over or near the spectral waveform on the display to indicate the characterization of the spectral waveform. Alternatively or additionally, a color of the spectral waveform may be indicative of the classification, e.g., the spectral waveform may be depicted on the display in red if characterized as monophasic, the spectral waveform may be depicted on the display in blue if characterized as biphasic, or the spectral waveform may be depicted on the display in green if characterized as triphasic.

Returning to FIG. 1, the database 108 may store digital patient data, e.g., in the form of an electronic health record (EHR) or other file(s) for each patient. In some embodiments, the mobile computing device 104 communicates through the network 106 with the database 108 to provide data for inclusion in the EHRs or other files of patients. By way of example, the mobile computing device 104 may upload imaging data (e.g., B-mode images, Doppler components, and/or spectral waveforms) generated together with the probe 102 for a given patient 110 to an EHR or other digital file of the patient stored on the database 108.

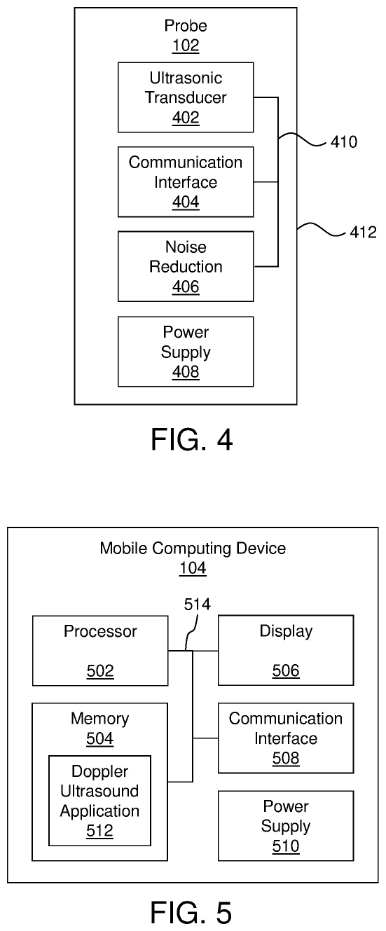

FIG. 4 illustrates an example implementation of the probe 102 of FIG. 1, arranged in accordance with at least one embodiment described herein. In general, the probe 102 may include one or more of an ultrasonic transducer 402, a communication interface 404, noise reduction 406, and/or a power supply 408. Although not illustrated in FIG. 4, the probe 102 may further include a processor, memory, and/or other components.

The ultrasonic transducer 402 may include a piezoelectric transducer, a capacitive micromachined ultrasonic transducer, or other suitable transducer. Although a single ultrasonic transducer 402 is depicted in FIG. 4, more generally the probe 102 may include one or more ultrasonic transducers, such as a linear or 2D array of ultrasonic transducers. The ultrasonic transducer 402 may include a tunable ultrasonic transducer configured to emit ultrasonic signals with different frequencies at different times, such as ultrasonic signals with a first frequency at a first time and ultrasonic signals with a second frequency at a second time.

The communication interface 404 includes any suitable interface for communicating wirelessly with other devices, such as with the mobile computing devices 104. For example, the communication interface 404 may include a Bluetooth communication interface, an 802.11-compliant communication interface, or other suitable wireless communication interface.

The noise reduction module 406 may include one or more circuits, programs, or other hardware or software to digitally reduce noise and improve sound quality, e.g., of electrical signals generated by the transducer 402 responsive to receiving reflected ultrasound signals.

The power supply 408 may include a mobile power supply, such as a battery, to supply power to the ultrasonic transducer 402, the communication interface 404, the noise reduction module 406, and/or other components of the probe 102 (such as a processor and/or memory).

The probe 102 may further include a communication bus 410 over which the various components of the probe 102 may communicate with each other. The communication bus 410 may include an interface bus, a memory bus, a storage interface bus, and/or other suitable communication bus.

In an example implementation, the compact Doppler ultrasound probe 102 of FIG. 4 includes the ultrasonic transducer 402, the wireless communication interface 404, and the power supply 408 coupled to the ultrasonic transducer 402 and the wireless communication interface 404 to supply power thereto. The ultrasonic transducer 402 may be configured to generate and emit ultrasound signals into a patient, receive reflected ultrasound signals from the patient, and convert the reflected ultrasound signals into electrical signals. The wireless communication interface 404 may be configured to wirelessly transmit the electrical signals to a mobile computing device, such as the mobile computing device 104, for processing into Doppler ultrasound data that includes blood flow velocity, e.g., Doppler mode components, of a blood vessel of the patient. Alternatively or additionally, the mobile computing device 104 may process the electrical signals received from the probe 102 into B-mode images, spectral waveforms, and/or other ultrasound data.

The probe 102 may further include a housing 412 within which each of the ultrasonic transducer 402, the wireless communication interface 404, the noise reduction module 406, and/or the power supply 408 is at least partially enclosed. The housing 412 may be about the size of a pencil. In these and other embodiments, a length of the housing may be in a range from 5 cm to 20 cm. Alternatively or additionally, an outer perimeter of the housing 412 may fit within a rectangular area arranged perpendicular to the length of the housing, the rectangular area having a first side and a second side perpendicular to the first side, each of the first and second sides having a length in a range from 0.3 cm to 2 cm. Alternatively or additionally, the length of each of the first and second sides of the rectangular area may be in a range from 0.5 cm to 0.9 cm.

FIG. 5 illustrates an example implementation of the mobile computing device 104 of FIG. 1, arranged in accordance with at least one embodiment described herein. In general, the mobile computing device 104 may include one or more of a processor 502, a memory 504, a display 506, a communication interface 508, and/or a power supply 510.

The processor 502 may be of any type such as a central processing unit (CPU), a microprocessor (μP), a microcontroller (μC), a digital signal processor (DSP), or any combination thereof. The processor 502 may be configured to execute computer instructions that, when executed, cause the processor 502 to perform or control performance of one or more of the operations described herein with respect to the mobile computing device 104.

The memory 504 may more generally include a storage medium, specifically any non-transitory computer-readable medium, including volatile memory such as random access memory (RAM), persistent or non-volatile storage such as read only memory (ROM), electrically erasable and programmable ROM (EEPROM), compact disc-ROM (CD-ROM) or other optical disk storage, magnetic disk storage or other magnetic storage device, NAND flash memory or other solid state storage device, or other persistent or non-volatile computer storage medium. The memory 504 may store computer instructions, such as a Doppler ultrasound application 512, that may be executed by the processor 502 to perform or control performance of one or more of the operations described herein with respect to the mobile computing device 104.

The display 506 may include a touchscreen display, a non-touchscreen display, or any other suitable display. Although not depicted in FIG. 5, the mobile computing device 104 may further include or be coupled to one or more input devices, such as a touchscreen, a keyboard, a stylus, a mouse, or other suitable input device. The input device(s) may be operated to configure operation of the probe 102 via the Doppler ultrasound application 512, such as by tuning the ultrasonic transducer 402 to a desired frequency.

The communication interface 508 includes any suitable interface for communicating wirelessly with other devices, such as with the probe 102. For example, the communication interface 404 may include a Bluetooth communication interface, an 802.11-compliant communication interface, or other suitable wireless communication interface.

The power supply 510 may include a mobile power supply, such as a battery, to supply power to the processor 502, the memory 504, the display 506, the communication interface 508, and/or other components of the mobile computing device.

The mobile computing device 104 may further include a communication bus 514 over which the various components of the mobile computing device 104 may communicate with each other. The communication bus 514 may include an interface bus, a memory bus, a storage interface bus, and/or other suitable communication bus.

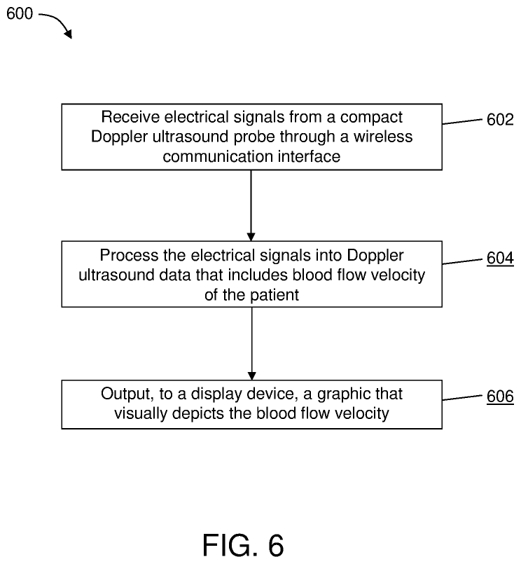

FIG. 6 illustrates a flowchart of an example method 600 to use an ultrasound imaging system, arranged in accordance with at least one embodiment described herein. The ultrasound imaging system may include the ultrasound imaging system of claim 1, including the probe 102 and the Doppler ultrasound computer program (e.g., Doppler ultrasound application 512 in FIG. 5). The method 600 may be performed by any suitable system, apparatus, or device. For example, any one or more of the mobile computing devices 104 described herein may perform or direct performance of one or more of the operations associated with the method 600. In these and other embodiments, the method 600 may be performed or controlled by one or more processors based on one or more computer-readable instructions stored on one or more non-transitory computer-readable media. Alternatively or additionally, embodiments herein may include a non-transitory computer-readable medium having computer-readable instructions stored thereon that are executable by a processor to perform or control performance of the method 600 or one or more operations thereof. The method 600 may include one or more of blocks 602, 604, and/or 606.

At block 602, the method 600 includes receiving electrical signals from a compact Doppler ultrasound probe through a wireless communication interface of the compact Doppler ultrasound probe. The compact Doppler ultrasound probe and the wireless communication interface may respectively include the probe 102 and the wireless communication interface 404 described herein. Block 602 may be followed by block 604.

At block 604, the method 600 may include processing the electrical signals into Doppler ultrasound data that includes blood flow velocity of the patient. Block 604 may be followed by block 606.

At block 606, the method 600 may include outputting, to a display device, a graphic that visually depicts the blood flow velocity. For example, the blood flow velocity may include a direction and/or numerical value or other information, some or all of which may be displayed as a graphic, e.g., on the display 506 of the mobile computing device 104.

One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments. Moreover, the method 600 may be combined with one or more other methods and processes described herein and/or steps or operations of the method 600 may be substituted for steps or operations of one or more other methods and processes described herein, or vice versa.

In some embodiments, the blood flow velocity is visually depicted in the graphic as a time-varying spectral waveform. In this and other embodiments, the method 600 may further include characterizing the time-varying spectral waveform as one of monophasic, biphasic, or triphasic. Alternatively or additionally, the method 600 may further include visually depicting the characterization on the display device. For example, visually depicting the characterization on the display device may include one or more of: depicting the time-varying spectral waveform in a first color (e.g., red) if the time-varying spectral waveform is characterized as monophasic; depicting the time-varying spectral waveform in a second color (e.g., blue) if the time-varying spectral waveform is characterized as biphasic; or depicting the time-varying spectral waveform in a third color (e.g., green) if the time-varying spectral waveform is characterized as triphasic.

In some embodiments, the method 600 may include saving at least one of the Doppler ultrasound data or the graphic to an EHR or other digital file of the patient.

In some embodiments, the method 600 includes one or more steps performed or controlled by the probe 102. For example, the method 600 may include generating and emitting ultrasound signals into a patient, receiving reflected ultrasound signals from the patient, and converting the reflected ultrasound signals into electrical signals. In these and other embodiments, the probe may be tunable and the method 600 may include receiving input to change a frequency of the ultrasound signals generated from a first frequency to a different second frequency. The input may be received through an app or other computer program (e.g., Doppler ultrasound application 512) executing on a mobile computing device having the display to which the graphic is output. The method 600 may include tuning the ultrasonic transducer to generate ultrasound signals at the second frequency. The method 600 may include emitting ultrasound signals at the second frequency.

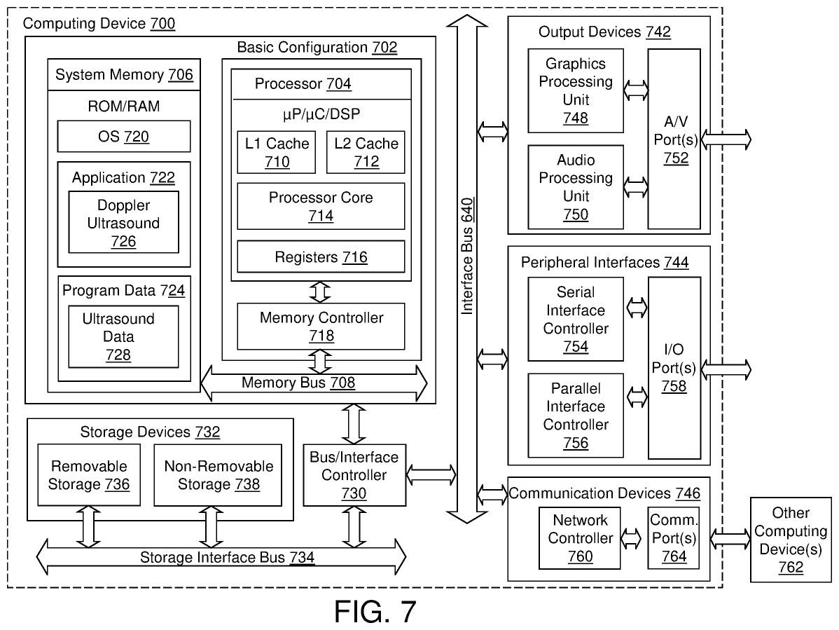

FIG. 7 is a block diagram illustrating an example computing device 700 that is arranged to process and display ultrasound data, arranged in accordance with at least one embodiment described herein. The computing device 700 may include, be included in, or otherwise correspond to either or both of the probe 102 or the mobile computing devices 104 described herein. In a basic configuration 702, the computing device 700 typically includes one or more processors 704 and a system memory 706. A memory bus 708 may be used to communicate between the processor 704 and the system memory 706.

Depending on the desired configuration, the processor 704 may be of any type including, but not limited to, a microprocessor (μP), a microcontroller (μC), a digital signal processor (DSP), or any combination thereof. The processor 704 may include one or more levels of caching, such as a level one cache 710 and a level two cache 712, a processor core 714, and registers 716. The processor core 714 may include an arithmetic logic unit (ALU), a floating point unit (FPU), a digital signal processing core (DSP Core), or any combination thereof. An example memory controller 718 may also be used with the processor 704, or in some implementations the memory controller 718 may include an internal part of the processor 704.

Depending on the desired configuration, the system memory 706 may be of any type including volatile memory (such as RAM), nonvolatile memory (such as ROM, flash memory, etc.), or any combination thereof. The system memory 706 may include an operating system 720, one or more applications 722, and program data 724. The application 722 may include a Doppler ultrasound application 726 that is arranged to process and display ultrasound data, including B-mode images, Doppler mode components, spectral waveforms, and/or other ultrasound data. The Doppler ultrasound application 726 may include, be included in, or otherwise correspond to the Doppler ultrasound application 512 of FIG. 5. The program data 724 may include ultrasound data 728 (which may include the raw data (e.g., electrical signals that represent reflected ultrasound signals) received from a probe, or data derived therefrom such as B-mode images, Doppler components, and/or spectral waveforms). In some embodiments, the application 722 may be arranged to operate with the program data 724 on the operating system 720 such that one or more methods or operations may be provided as described herein.

The computing device 700 may have additional features or functionality, and additional interfaces to facilitate communications between the basic configuration 702 and any involved devices and interfaces. For example, a bus/interface controller 730 may be used to facilitate communications between the basic configuration 702 and one or more data storage devices 732 via a storage interface bus 734. The data storage devices 732 may be removable storage devices 736, non-removable storage devices 738, or a combination thereof. Examples of removable storage and non-removable storage devices include magnetic disk devices such as flexible disk drives and hard-disk drives (HDDs), optical disk drives such as compact disk (CD) drives or digital versatile disk (DVD) drives, solid state drives (SSDs), and tape drives to name a few. Example computer storage media may include volatile and nonvolatile, removable and non-removable media implemented in any method or technology for storage of information, such as computer-readable instructions, data structures, program modules, or other data.

The system memory 706, the removable storage devices 736, and the non-removable storage devices 738 are examples of computer storage media or non-transitory computer-readable media. Computer storage media or non-transitory computer-readable media includes RAM, ROM, EEPROM, flash memory or other memory technology, CD-ROM, digital versatile disks (DVDs) or other optical storage, magnetic cassettes, magnetic tape, magnetic disk storage or other magnetic storage devices, or any other non-transitory medium which may be used to store the desired information and which may be accessed by the computing device 700. Any such computer storage media or non-transitory computer-readable media may be part of the computing device 700.

The computing device 700 may also include an interface bus 740 to facilitate communication from various interface devices (e.g., output devices 742, peripheral interfaces 744, and communication devices 746) to the basic configuration 702 via the bus/interface controller 730. The output devices 742 include a graphics processing unit 748 and an audio processing unit 750, which may be configured to communicate to various external devices such as a display or speakers via one or more A/V ports 752. B-mode images, Doppler components, spectral waveforms, and/or other ultrasound data generated by the Doppler ultrasound application 726 may be output through the graphics processing unit 748 to such a display. The peripheral interfaces 744 include a serial interface controller 754 or a parallel interface controller 756, which may be configured to communicate with external devices such as input devices (e.g., keyboard, mouse, pen, voice input device, touch input device, etc.), sensors, or other peripheral devices (e.g., printer, scanner, etc.) via one or more I/O ports 758. Such input devices may be operated by a user to provide input to the Doppler ultrasound application 726, which input may be effective to, e.g., tune an ultrasonic transducer to a desired frequency, save ultrasound data to a patient's EHR or other digital file, and/or to accomplish other operations via the Doppler ultrasound application 726. The communication devices 746 include a network controller 760, which may be arranged to facilitate communications with one or more other computing devices 762 over a network communication link via one or more communication ports 764.

The network communication link may be one example of a communication media. Communication media may typically be embodied by computer-readable instructions, data structures, program modules, or other data in a modulated data signal, such as a carrier wave or other transport mechanism, and may include any information delivery media. A “modulated data signal” may be a signal that has one or more of its characteristics set or changed in such a manner as to encode information in the signal. By way of example, and not limitation, communication media may include wired media such as a wired network or direct-wired connection, and wireless media such as acoustic, radio frequency (RF), microwave, infrared (IR), and other wireless media. The term “computer-readable media” as used herein may include both storage media and communication media.

The computing device 700 may be implemented as a portion of a small-form factor portable (or mobile) electronic device such as a smartphone, a personal data assistant (PDA) or an application-specific device. The computing device 700 may also be implemented as a personal computer including tablet computer, laptop computer, and/or non-laptop computer configurations, or a server computer including both rack-mounted server computer and blade server computer configurations.

Unless specific arrangements described herein are mutually exclusive with one another, the various implementations described herein can be combined to enhance system functionality or to produce complementary functions. Likewise, aspects of the implementations may be implemented in standalone arrangements. Thus, the above description has been given by way of example only and modification in detail may be made within the scope of the present invention.

With respect to the use of substantially any plural or singular terms herein, those having skill in the art can translate from the plural to the singular or from the singular to the plural as is appropriate to the context or application. The various singular/plural permutations may be expressly set forth herein for sake of clarity. A reference to an element in the singular is not intended to mean “one and only one” unless specifically stated, but rather “one or more.” Moreover, nothing disclosed herein is intended to be dedicated to the public regardless of whether such disclosure is explicitly recited in the above description.

In general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). Furthermore, in those instances where a convention analogous to “at least one of A, B, and C, etc.” is used, in general, such a construction is intended in the sense one having skill in the art would understand the convention (e.g., “a system having at least one of A, B, and C” would include but not be limited to systems that include A alone, B alone, C alone, A and B together, A and C together, B and C together, or A, B, and C together, etc.). Also, a phrase presenting two or more alternative terms, whether in the description, claims, or drawings, should be understood to include one of the terms, either of the terms, or both terms. For example, the phrase “A or B” will be understood to include the possibilities of “A” or “B” or “A and B.”

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. A compact Doppler ultrasound probe comprising;
an ultrasonic transducer;
a wireless communication interface; and
a power supply coupled to the ultrasonic transducer and the wireless communication interface;
wherein the ultrasonic transducer is configured to generate and emit ultrasound signals into a patient, receive reflected ultrasound signals from the patient, and convert the reflected ultrasound signals into electrical signals.

2. The compact Doppler ultrasound probe of claim 3, wherein the wireless communication interface is configured to wirelessly transmit the electrical signals to a mobile computing device for processing into Doppler ultrasound data that includes blood flow velocity of a blood vessel of the patient.

3. The compact Doppler ultrasound probe of claim 1, further comprising a housing within which each of the ultrasonic transducer, the wireless communication interface, and the power supply is at least partially enclosed, the housing being about the size of a pencil.

4. The compact Doppler ultrasound probe of claim 1, wherein a length of the housing is in a range from 5 centimeters (cm) to 20 cm.

5. The compact Doppler ultrasound probe of claim 4, wherein along the length of the housing, an outer perimeter of the housing fits within a rectangular area arranged perpendicular to the length of the housing, the rectangular area having a first side and a second side perpendicular to the first side, each of the first and second sides having a length in a range from 0.3 cm to 2 cm.

6. The compact Doppler ultrasound probe of claim 5, wherein the length of each of the first and second sides is in a range from 0.5 cm to 0.9 cm.

7. The compact Doppler ultrasound probe of claim 1, wherein the ultrasonic transducer comprises a tunable ultrasonic transducer.

8. The compact Doppler ultrasound probe of claim 1, wherein the wireless communication interface comprises at least one of a Bluetooth communication interface or an 802.11-compliant communication interface.

9. An ultrasound imaging system, comprising:
a compact Doppler ultrasound probe including an ultrasonic transducer, a wireless communication interface, and a power supply coupled to the ultrasonic transducer and the wireless communication interface, wherein the ultrasonic transducer is configured to generate and emit ultrasound signals into a patient, receive reflected ultrasound signals from the patient, and convert the reflected ultrasound signals into electrical signals; and
a computer-readable medium having computer-readable instructions stored thereon, the computer-readable instructions executable by a processor to perform or control performance of operations comprising:
receiving the electrical signals from the compact Doppler ultrasound probe through the wireless communication interface;
processing the electrical signals into Doppler ultrasound data that includes blood flow velocity of the patient; and
outputting, to a display device, a graphic that visually depicts the blood flow velocity.

10. The ultrasound imaging system of claim 9, wherein the blood flow velocity is depicted as a time-varying spectral waveform, and the operations further comprise:
characterizing the time-varying spectral waveform as one of monophasic, biphasic, or triphasic; and
visually depicting the characterization on the display device.

11. The ultrasound imaging system of claim 10, wherein visually depicting the characterization on the display device includes one of:
depicting the time-varying spectral waveform in red if the time-varying spectral waveform is characterized as monophasic;
depicting the time-varying spectral waveform in blue if the time-varying spectral waveform is characterized as biphasic; or
depicting the time-varying spectral waveform in green if the time-varying spectral waveform is characterized as triphasic.

12. The ultrasound imaging system of claim 9, the operations further comprising saving at least one of the Doppler ultrasound data or the graphic to an Electronic Health Record (EHR) of the patient.

13. The ultrasound imaging system of claim 9, wherein the compact Doppler ultrasound probe is about the size of a pencil.

14. The ultrasound imaging system of claim 9, wherein the ultrasonic transducer comprises a tunable ultrasonic transducer.

15. The ultrasound imaging system of claim 14, the operations further comprising:
receiving input to change a frequency of the generated ultrasound signals from a first frequency to a different second frequency;
tuning the ultrasonic transducer to generate ultrasound signals at the second frequency; and
emitting ultrasound signals at the second frequency.

16. A method, comprising:
generating and emitting ultrasound signals into a patient from a compact Doppler ultrasound probe;
receiving reflected ultrasound signals from the patient at the compact Doppler ultrasound probe;
converting the reflected ultrasound signals into electrical signals; and
transmitting the electrical signals wirelessly to a mobile computing device for processing into Doppler ultrasound data.

17. The method of claim 16, wherein the emitted ultrasound signals have a first frequency, the method further comprising:
tuning an ultrasonic transducer of the compact Doppler ultrasound probe to generate ultrasound signals at a second frequency; and
emitting ultrasound signals at the second frequency.

18. The method of claim 16, further comprising:
receiving the electrical signals wirelessly from the compact Doppler ultrasound probe at a mobile computing device;
processing the electrical signals into Doppler ultrasound data that includes blood flow velocity of the patient; and
outputting, to a display device of the mobile computing device, a graphic that visually depicts the blood flow velocity.

19. The method of claim 18, wherein the blood flow velocity is depicted as a time-varying spectral waveform, the method further comprising:
characterizing the time-varying spectral waveform as one of monophasic, biphasic, or triphasic; and
visually depicting the characterization on the display device.

20. The method of claim 19, wherein visually depicting the characterization on the display device includes one of:
depicting the time-varying spectral waveform in a first color if the time-varying spectral waveform is characterized as monophasic;
depicting the time-varying spectral waveform in a second color if the time-varying spectral waveform is characterized as biphasic; or
depicting the time-varying spectral waveform in a third color if the time-varying spectral waveform is characterized as triphasic.

21. The method of claim 18, further comprising saving at least one of the Doppler ultrasound data or the graphic to an Electronic Health Record (EHR) of the patient.
